(19) United States
(12) Patent Application Publication
(10) Pub. No.: US 2015/0034776 A1
(43) Pub. Date: Feb. 5, 2015

(54) LINE MANAGEMENT DEVICE

(71) Applicant: Stryker Corporation, Kalamazoo, MI (US)

(72) Inventors: Connor Feldpausch St.John, Marne, MI (US); Cory Patrick Herbst, Shelbyville, MI (US); Mark Jonathan Sherman, Hudsonville, MI (US); Kelly Ann Stieler, Kernersville, NC (US); Justin Lee Benacquisto, Algonac, MI (US)

(21) Appl. No.: 14/448,064

(22) Filed: Jul. 31, 2014

Related U.S. Application Data
(60) Provisional application No. 61/860,859, filed on Jul. 31, 2013.

(51) Int. Cl. A61M 5/14; F16L 3/23; F16L 3/02

(52) U.S. Cl. CPC A61M 5/1418; F16L 3/02; F16L 3/23
USPC 248/68.1

(57) ABSTRACT

A line management device includes a body with a slot for receiving a tube and a base adapted to mount the body to a support surface. The base is configured to adapt to a non-planar surface without affecting the shape of the slot.

This application claims the benefit of provisional application entitled LINE MANAGEMENT DEVICE, filed Jul. 31, 2013, U.S. Ser. No. 61/860,859.

### TECHNICAL FIELD AND BACKGROUND OF THE INVENTION

The present invention relates to line management device, and more particularly to a line management device that can hold tubing, namely tubing used during medical treatment and/or patient monitoring.

Current line management devices are formed from generally inelastic material, such as plastics or resins, which can break overtime and, in some cases, can create a pinch point for a caregiver when inserting a tube into the device. The devices also inherently pose a cleaning challenge due to its multiple and tight surfaces.

### SUMMARY OF THE INVENTION

Accordingly, a line management device is provided that includes a body with one or more slots for receiving tubing and which is configured to deflect when subject to a side load or deform when mounted to a curved surface but without affecting the shape of the slots.

In one form, a line management device includes a body with one or more slots extending into the body. The body includes a base, which is adapted to releasably mount the device to a support surface. The base is configured to adapt to a non-planar surface without affecting the shape of the slot.

In another form, a line management device includes a body with one or more slots extending into the body. The body extends from a base, which is adapted to releasably mount the device to a support surface. The body and base are configured to allow the body to deflect relative to the base but without affecting the shape of the slot.

In any of the above, the body includes a plurality of slots. Further, the size of the slots may vary in length and/or width. In yet a further aspect, one or more of the slots may be color coded for a specific use.

In any of the above, the body and the base may be monolithic. For example, the body and the base made be formed from a foam, such as cross-linked polyethylene foam or a urethane foam and further can be molded, such as cast molding or injection molding. Alternately, the body and the base may be formed together as a hollow member from a thin polymer material, such as plastic. The density of the foam may vary, for example, in a range of about 1-6 lbs./ft³, of about 2-5 lbs./ft³, or about 3-4 lbs./ft³.

In any of the above, the body and the base may have an antimicrobial component, such as by a coating, such as MICROBAN or by incorporating antimicrobial components, such as antimicrobial powder, antimicrobial bodies, antimicrobial elements, such as wires, etc. into the body and/or base during the forming process. Suitable antimicrobial component may be formed from silver, copper, or their alloys or the like.

In any of the above, the body may have a generally cylindrical shape with a curved outer surface and a central axis extending generally parallel to the curved outer surface, with the slots extending into the curved outer surface.

In any of the above, the body may have a thickness in a range of about ¾″ to 2″, or any dimension in between, including about 1″ or about 1½″.

In any of the above, the device may have a height in a range of about 1½″ to 3½″, or any dimension in between including in a range of about 2″ to 3″ inches, or about 2½″.

In any of the above, the body may have a generally cylindrical shape with a curved outer surface and a central axis extending generally parallel to the curved outer surface, with the slots extending into the curved outer surface.

In any of the above, the base may comprise a generally planar member. For example, the base may have a thickness in a range of ⅛″ to ½″, or any dimension in between including in a range of about 3/16″ to ⅜″ or about ¼″.

In any of the above, the base may have a length in a range of 1″ to 3″, or any dimension in between including in a range of about 1½″ to 2½″ or about 2″.

In any of the above, the base may have a width in a range of about ¾″ to 2″, or any dimension in between, including about 1″ or about 1½″.

In any of the above, the base may have an adhesive layer to mount the device to a support surface. For example, the adhesive surface may be provided by a double-sided adhesive tape or the adhesive may be applied to the base with a release paper over the adhesive.

Before the embodiments of the invention are explained in more detail below, it is to be understood that the invention is not limited to the details of operation or to the details of construction and the arrangement of the components set forth in the following description or illustrated in the drawings. The invention may be implemented in various other embodiments and is capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a perspective view of a line management device;

FIG. 2 is a top plan view of the line management device of FIG. 1;

FIG. 3 is a bottom plan view of the line management device of FIG. 1;

FIG. 4 is a right side view of the line management device of FIG. 1, which is a mirror image of the left side view;

FIG. 5 is a front elevation view of the line management device of FIG. 1 which is a mirror image of the rear elevation view;

FIG. 6 is a perspective view of another embodiment of a line management device;

FIG. 6A is another perspective view of the line management device of FIG. 6 showing shading;

FIG. 7 is a top plan view of the line management device of FIG. 6;

FIG. 8 is a bottom plan view of the line management device of FIG. 6;

FIG. 9 is a right side view of the line management device of FIG. 6 which is a mirror image of the left side view; and

FIG. 10 is a front elevation view of the line management device of FIG. 6, which is a mirror image of the rear elevation view;

FIG. 11 is perspective view another embodiment of a line management device;

FIG. 12 is another perspective view of the line management device of FIG. 11;

FIG. 13 is a perspective view of another embodiment of a line management device;

FIG. 14 is another perspective view of the line management device of FIG. 13;

FIG. 15 is another perspective view of the line management device of FIG. 11 without shading;

FIG. 16 is a top plan view of the line management device of FIG. 15;

FIG. 17 is a side view of the line management device of FIG. 16;

FIG. 18 is an end view of the line management device of FIG. 16;

FIG. 19 is another perspective view of the line management device of FIG. 13 without shading;

FIG. 20 is a top plan view of the line management device of FIG. 19;

FIG. 21 is a side view of the line management device of FIG. 19;

FIG. 22 is a perspective view of another embodiment of a line management device;

FIG. 23 is a top plan view of the line management device of FIG. 22; and

FIG. 24 is a cross-section view through line XXIV-XXV of FIG. 23.

### DETAILED DESCRIPTION OF THE PREFERRED EMBODIMENTS

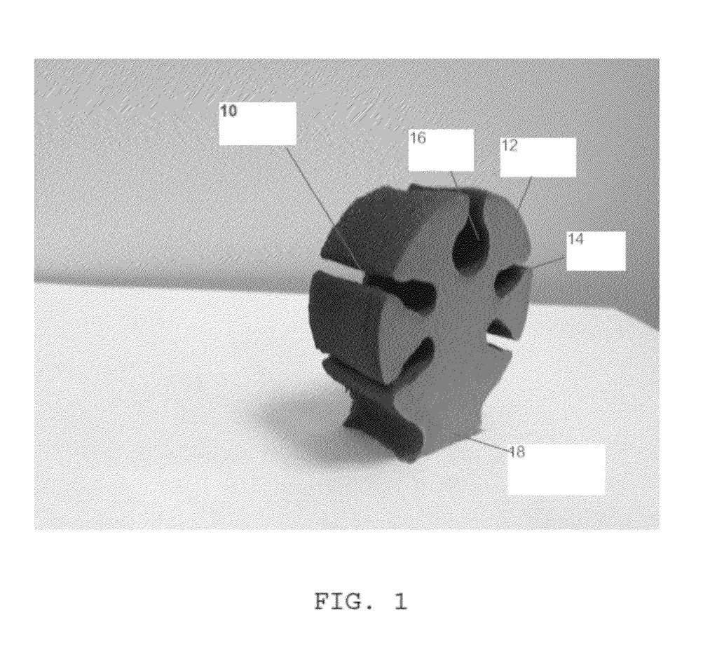

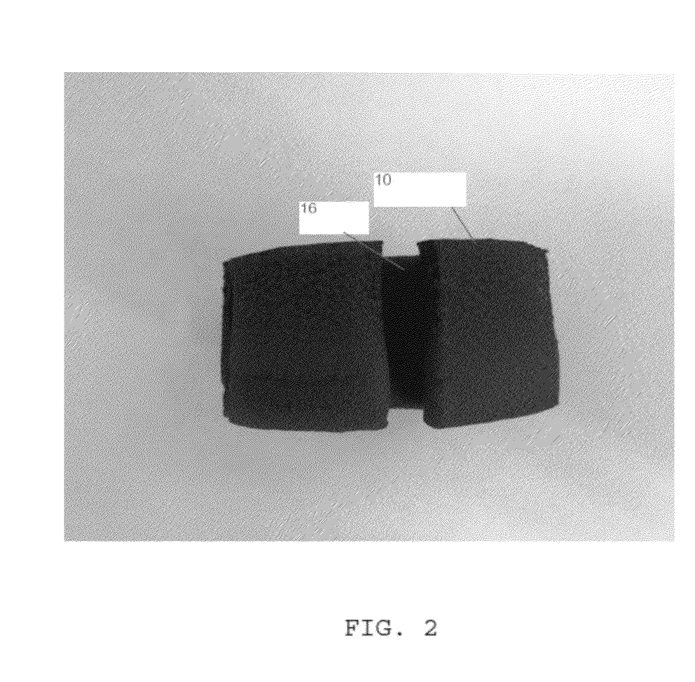

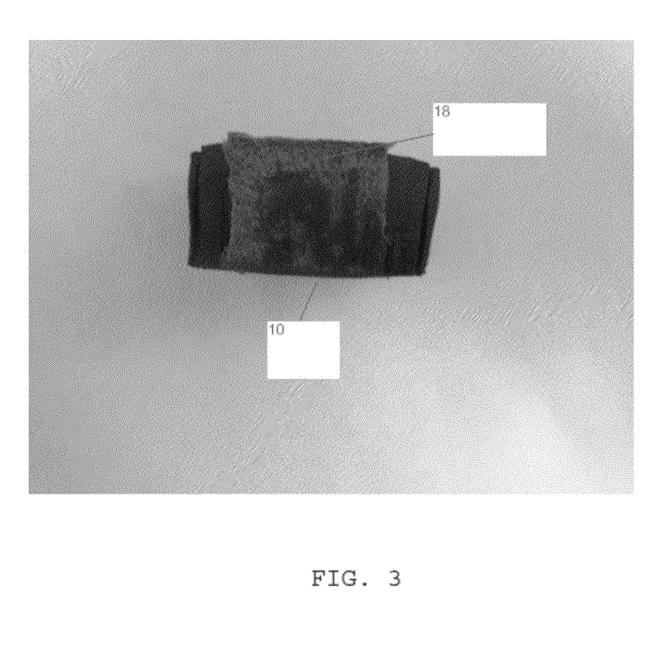

Referring to FIG. 1, the numeral 10 generally designates one embodiment of a line management device. As will be more fully described below, line management device 10 is formed with one or more slots for receiving one or more tubing and to have flexible and for compressible properties so that it can deflect or flex and/or compress in response to being knocked by a caregiver, for example, so that it does not create a hard touch point for the caregiver and potentially injure the caregiver. Further, because of its compressive properties, the device does not generally create any potential pinch points, for example, when a caregiver is trying to insert tubing into the device. In addition, the device is configured so that when it is deflected or flexed, the slots do not change their shape and continue to retain the tubing positioned therein.

As best seen in FIG. 1, device 10 includes a body 12 with one or more slots 14, 16, extending into the body, which body is formed from a compressible material to facilitate insertion and removal of the tubing into or from the slots. The number of the slots may vary. In addition, the slots may vary in length and/or width. Further as described below, one or more of the slots may be color coded to designate a specific use.

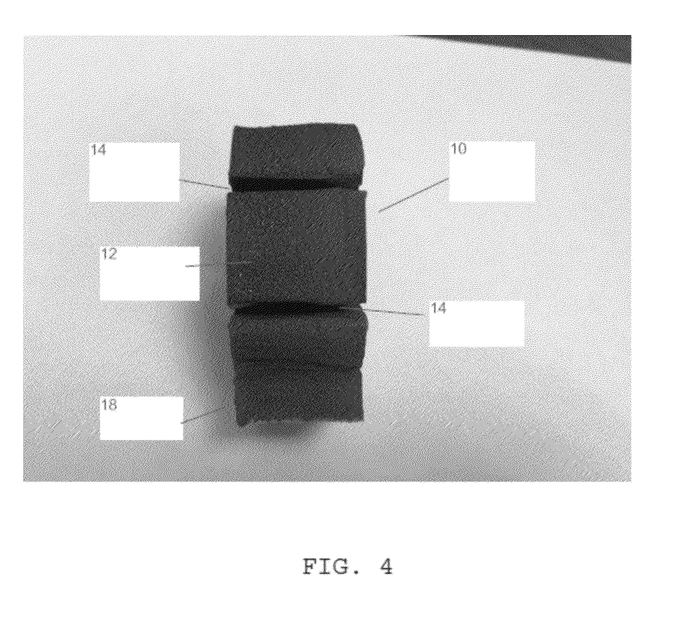

Referring to FIG. 5, slots 14 and 16 each have a narrower neck at the curved surface of the body and expand into a larger opening so that when tubing is inserted into the slot, the material on either side of the slot will either compress or deflect and diverge so that the tubing can be moved past the neck, with the sides of the material forming the neck returning to their uncompressed or un-deflected configuration to retain the tubing in the slot. For example, a caregiver may simple press the tubing into the neck of the slots, and the sides that define the slots will separate so that the tubing can be pressed so that it rests at the bottom of the slot. Once the tubing is in place, as noted, the sides will reform to their un-diverged configuration to thereby capture the tubing in the slot. Similarly, when the caregiver wishes to remove the tubing, the caregiver can simply pull on the tubing (e.g. on a section of tubing that is exiting the device and adjacent the device) which increases the size of the neck of the slot and allows the tubing to be removed from the device.

In the illustrated embodiment, body 12 includes multiple slots, including one slot 16 with a rounded end and with a width that is suitable for inserting and holding larger tubing, such as breathing liens or arterial lines. The other slots 14 are configured with a tear drop shape with a width and height to accommodate multiple, stacked smaller tubes, such as IV tubing. As would be understood, with multiple slots, the material between the slots can then form fingers that can easily flex and tilt about their base at the end of the slots in the direction of the unoccupied slot.

Device 10 also includes a base 18, which in the illustrated embodiment is integrally formed with body 12 so that the body and the base are monolithic. Device 10 optionally includes a tapered transition between the body and the base so base 18 is wedge shaped, and body 12 may deflect relative to base 18, for example when device is impacted by a load, such as when a caregiver bumps into the device. One result is that the body can deflect relative to the base but without affecting the shape of the slots.

For example, the body and the base may be molded as a unitary member and be formed from an elastic material, such as foam or structural gel. A suitable structure gel may include a SEB, SEBS, SEP, SEPS, SEEP, SEEPS polymer combined with a mineral oil, for example, as is commonly known in the gel art. A suitable foam includes a cross-linked polyethylene foam or a urethane foam. The density of the foam may vary, for example, in a range of about 1-6 lbs./ft³, of about 2-5 lbs./ft³, or about 3-4 lbs./ft³. In either case, the device can be molded, such as cast molding or injection molding. When formed from urethane foam, device may be formed with a conventional in-mold coating, which could include antimicrobial properties. Suitable antimicrobial coatings may include MICROBAN. During the forming process, the material surrounding the slots may be formed using a different color, as noted above, to provide color coding. Alternately, the color coding can be applied post forming.

Alternately, the body and the base may be formed, such as by molding, as a hollow member from a thin polymeric material, such as plastic. For example, the wall thickness may range from several thousands of an inch to tens of thousands of an inch, for example in a range of 10/1000^{th} of an inch to 50/1000^{th} of an inch so that the body and base can compress and deflect in a similar manner as described above.

Base 18 is adapted to releasably mount the device to a support surface, for example a hospital bed barrier, such a side rail, footboard, and/or headboard. For example, the base may have an adhesive layer, including a 2 sided adhesive tape, that releasably mounts the device to a support surface, such as a double-side adhesive tape or an adhesive applied to the base with a release paper over the adhesive. Alternately, the device may incorporate a COMMAND STRIP, available from 3M.

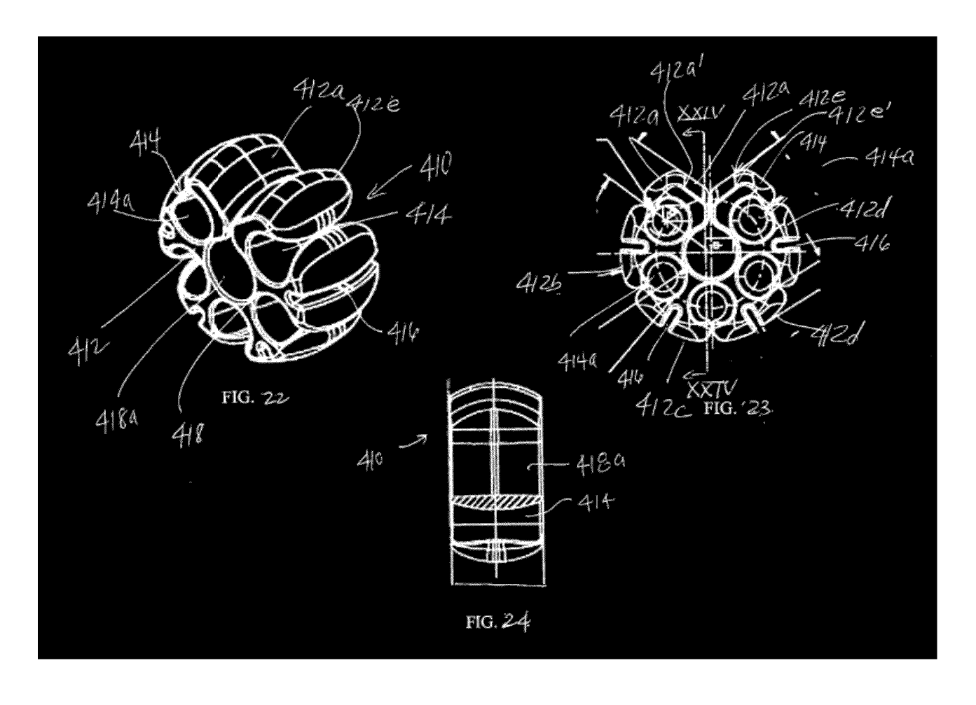

Further, the base may be configured to adapt to a non-planar surface, for example, to an IV pole or IV pole mount. For further details of another embodiment of the line management device that is formed as a pole mount version reference is made to FIGS. 22-24 described more fully below. Because the body can flex about the base, when the base is deformed to follow the surface topology of a curved mounting surface the slots will remain in their configuration so that the tubing will not be unintentionally released. Similarly, if a caregiver bumps into the device, the body can deflect without the slots changing shape. Additionally, because of the adaptability of the base, device 10 can be mounted on any bed and any surface.

As noted above, when forming device 10, device 10 may be covered with a coating that has antimicrobial properties. Alternately or in addition, the body and the base may have an antimicrobial component, such as by incorporating antimicrobial components, such as antimicrobial powder, antimicrobial bodies, antimicrobial elements, such as wires, etc. into the body and/or base during the forming process. Suitable antimicrobial component may be formed from silver, copper, or their alloys or the like.

Referring again to FIGS. 1 and 5, body 12 may have a generally cylindrical shape with a longitudinal axis 12a, a curved outer surface 12b, and a central axis 12c, which extends generally parallel to the curved outer surface and is orthogonal to longitudinal axis 12a. Slots 14 and 16 may extend radially inward through the curved outer surface toward the central axis. Similarly the material between the slots, which form fingers with enlarged ends (which form the shoulders on either side of the slots' necks), may also extend along radial axes that extend through central axis 12c.

To provide sufficient mass and structure to device 10, body 12 may have a thickness (as measured along axis 12c) in a range of about ¾″ to 2″, or any dimension in between, including about 1″ or about 1½″. Further, the device may have a height in a range of about 1½″ to 3½″, or any dimension in between including about in a range of about 2″ to 3″ inches, or about 2½″.

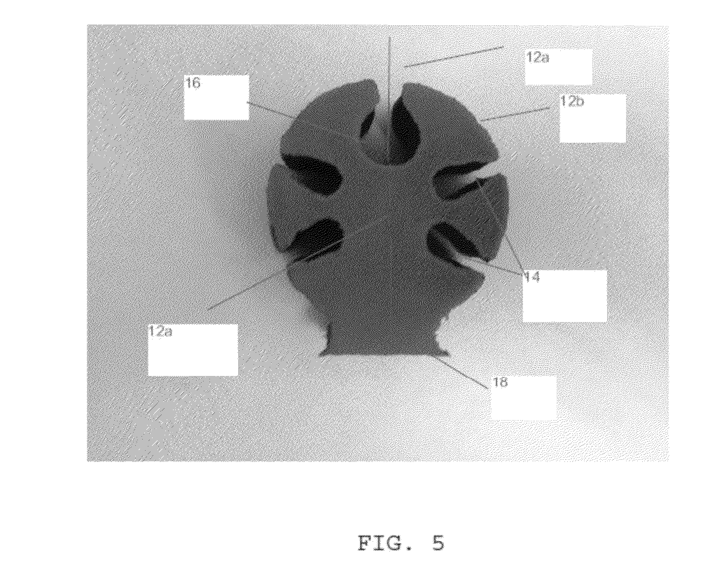

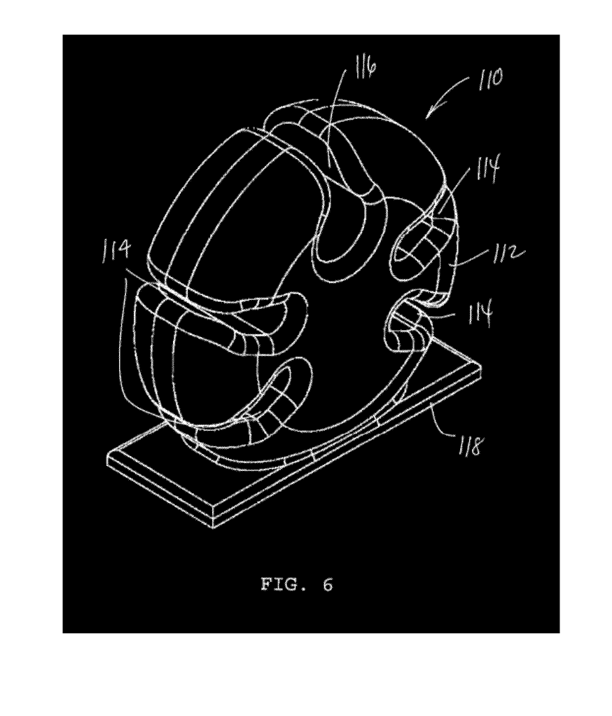

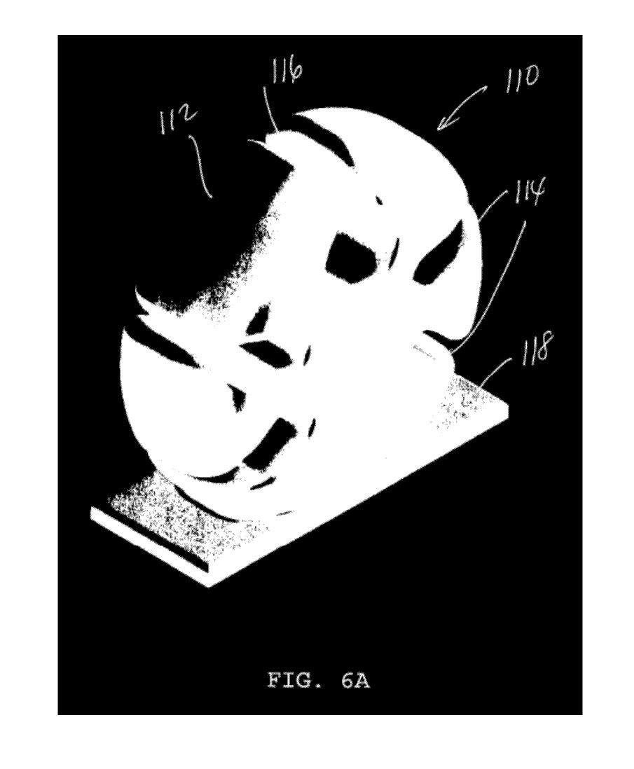

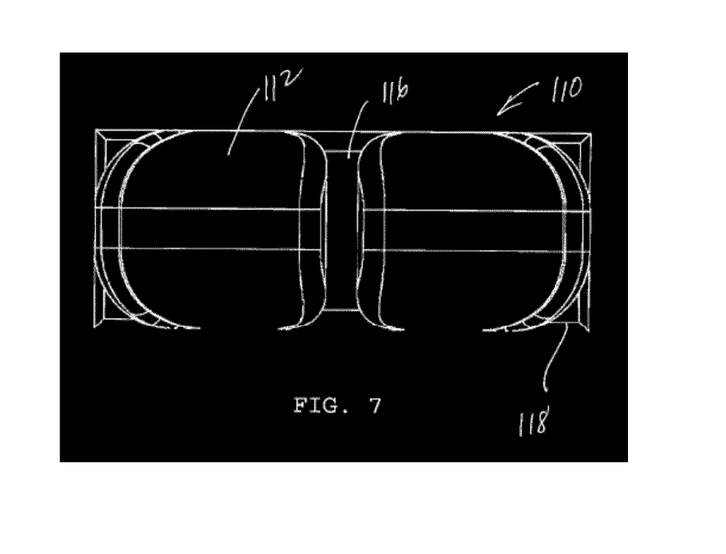

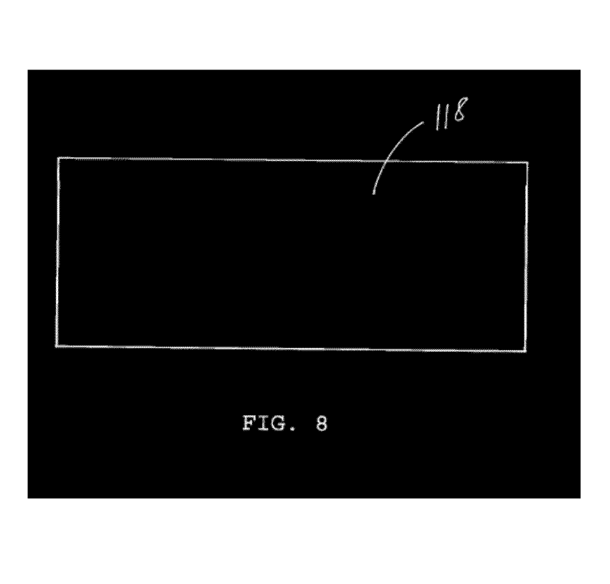

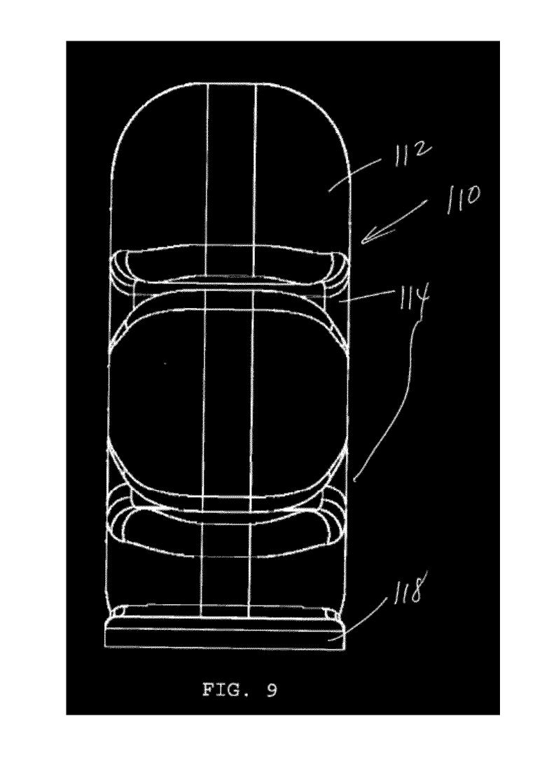

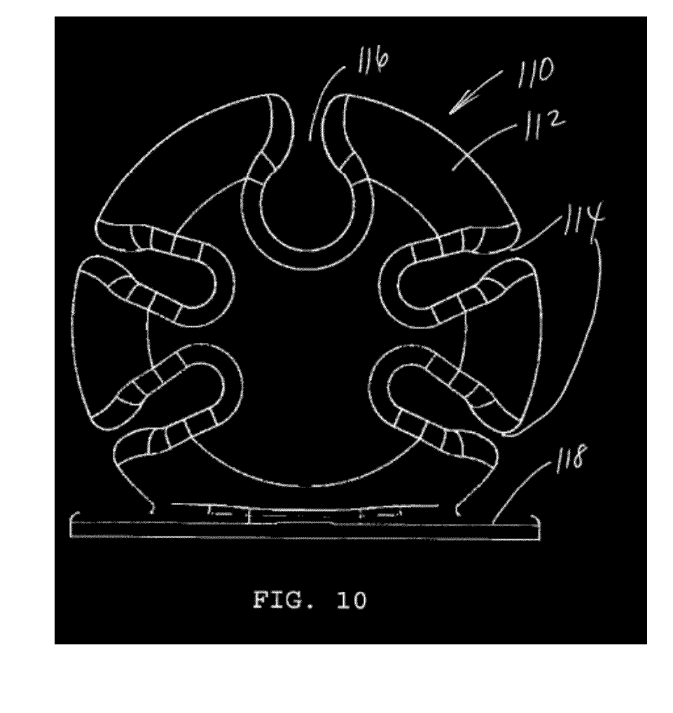

Referring to FIGS. 6-10, the numeral 110 generally designates another embodiment of a line management device. Device 110 is similar to device 10 except that it has rounded edges at its body 112 outer periphery and around the respective slots 114 and 116, and that it has an enlarged base 118.

As best seen FIG. 6, in the illustrated embodiment, base 118 is formed by planar member, which provides even greater flexibility to base 118. For example, the base may have a thickness in a range of ⅛″ to ½″, or any dimension in between including about in a range of about 3/16″ to ⅜″ or about ¼″. Further, the base may have a length in a range of 1″ to 3″, or any dimension in between including about in a range of about 1½″ to 2½″ or about 2″, and a width in a range of about ¾″ to 2″, or any dimension in between, including about 1″ or about 1½″. In this manner, base 118 can easily be mounted about an IV pole, again without impacting the shape of the slots.

For further details, for example, of the slots 114, 116, the material and method of forming device 110, reference is made above to device 10.

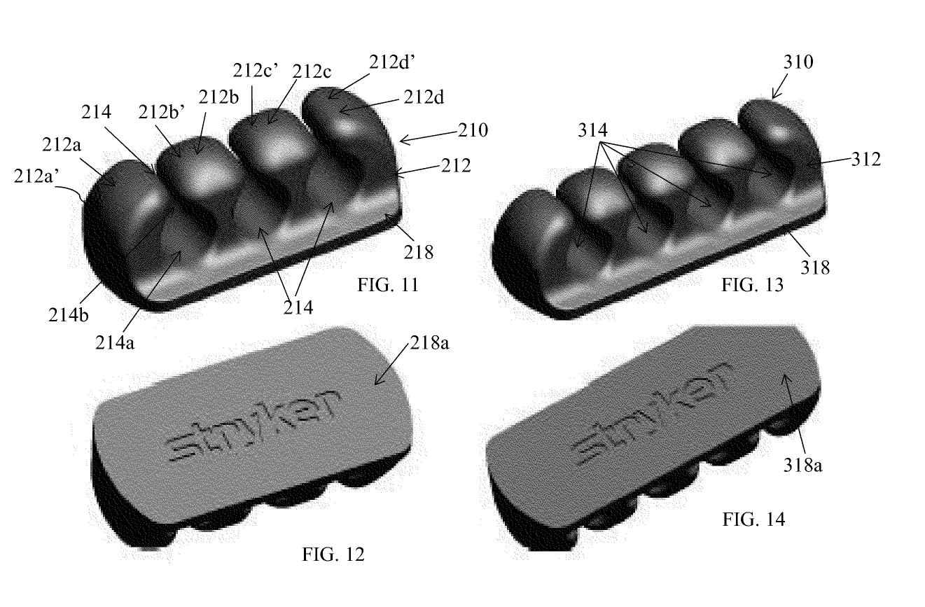

Referring to FIGS. 11 and 12 and 15-18, the numeral 210 generally designates another embodiment of a line management device. Device 210 includes a body 212 that forms a base 218 for mounting to a surface, such as those noted above, including a hospital bed. Device 210 similarly includes a plurality of slots 214 that extend into body 212 from an upper side, as viewed in FIG. 11. Slots 214 also extend through body 212 from one side of body 212 to the opposed side of body 212 and are adapted to support and hold tubes, such as IV tubing for delivering fluids or removing fluids from a patient or such as cables for transmitting electrical signals from monitoring devices. Similar to the previous embodiments, body 212 may be molded as a unitary member and be formed from an elastic material, such as foam or structural gel, so that body 212 is flexible and exhibits compressible properties so that it can deflect or flex and/or compress in response to being knocked by a caregiver. In this manner, device 210 will not create a hard touch point for the caregiver and potentially injure the caregiver. As best seen in FIG. 17, base 218 includes a generally planar surface 218a that forms a mounting surface for the device. As noted above in reference to the earlier embodiments, the base may include a layer of adhesive, including a 2-sided adhesive tape, for mounting the device to the desired surface. Extending from base 218 are body segments 212a, 212b, 212c, and 212d, which are formed by the material surrounding the respective slots. Each segment 212a, 212b, 212c, and 212d has an angled or curved outer surface 212a′, 212b′, 212c′, and 212d′, which can form cam surfaces to urge the respective segments to separate when tubing is pushed into the respective slots. Optionally the distal end of each segment may have a small planar region, which is surrounded by the curved or angled outer surface of the respective segment.

In the illustrated embodiment, slots 214 each include a generally cylindrical portion 214a and a narrowed neck 214b, which is formed between the respective adjacent surfaces of the adjacent segments. Optionally, the adjacent segments may be configured so that they have no gap or a very small gap (e.g. 0.02-0.06 inches) between them so that they touch or nearly touch, and, hence, the narrowed necks 214b could go down to zero width or close to zero width. Further, each slot 214 may have a longitudinal extent that extends into the body, with the longitudinal extents of each slot being substantially the same so that the depth or height of each slot in device 210 is about the same. Similarly each slot includes a lateral extent, which at its widest point is generally the same as the lateral extent of each adjacent slot so that the slots may have similar dimensions, though as described above and below the dimensions of the slots may vary. For example, the depth of the slots may be optionally in a range of about 0.5 to 1.5 inches or about 0.75 inches, with the diameter of the cylindrical portion of the slots optionally in a range of about 0.2 to 0.8 inches or about 0.5 inches.

The dimensions of the body of the device may also vary. For example, in the three slot version, device 210 may have a length in a range of about 3.0 to 4.0 inches or be in a range of about 3.5 to 3.75 inches. Device 210 may have a width in a range of about 1.0 to 2.0 inches or be in a range of about 1.25 to 1.75 inches. The height may range from about 0.75 to 1.50 inches or be in a range of about 1.0 to 1.25 inches. For a four slot version, noted below, the length may be increased.

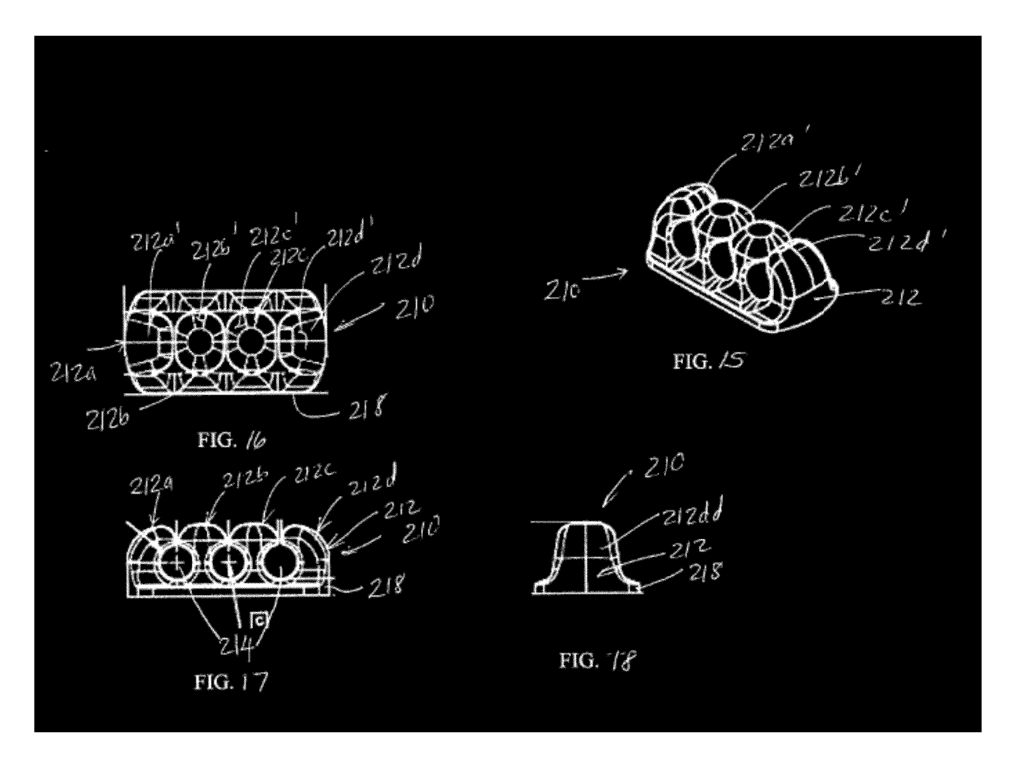

Referring to FIGS. 13 and 14 and 19-21, the numeral 310 designates yet another embodiment of a line management device. Line management device 310 is a similar construction to line management device 210 and includes a body 312 and a base 318 and further four slots 314. For further details of line management device 310 reference is made to line management device 210.

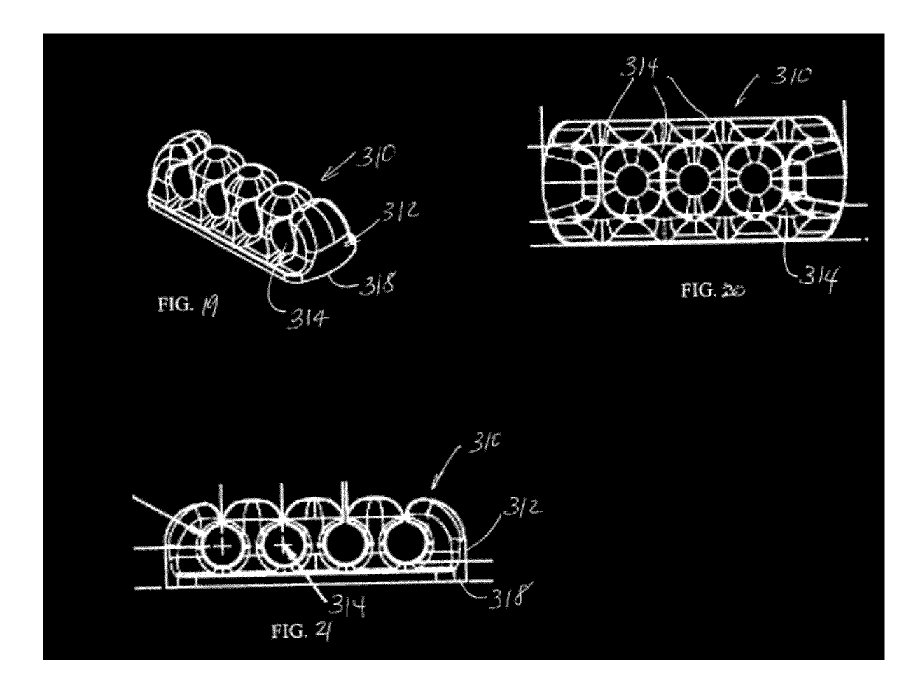

Referring to FIGS. 21-23, the numeral 410 designates another embodiment of a line management device. Line management device 410 similarly includes a body 412, which forms a base 418, and a plurality of slots 414 that extend into body 412 and are adapted to hold tubing therein. In the illustrated embodiment, line management device 410 is adapted to mount about a pole. To that end, base 418 comprises an annular base with a central opening 418a and with segments 412a 412b, 412c, 412d, and 412e, which are formed by the material surrounding the slots, extending radially outward from annular base 418.

Slots 414 are similar to slots 214 and 314 in that they each have cylindrical portion 414a and a narrowed neck 414b. Optionally, as shown in FIG. 24, cylindrical portions 414a each may be tapered and have a narrower cross section in their middle section than at their opposed ends. In this manner, when body 412 is formed from an elastomeric material, the tube or tubing when inserted into the respective slots may compress the adjacent segments to thereby be frictionally held within the respective slots.

Optionally, device 410 may include a second plurality of slots 416 that extend partially into each segment 412a 412b, 412c, 412d, and 412e to form smaller slots for holding smaller tubes or tubing. Slots 416 may have a rounded distal end but may be defined by generally parallel facing surfaces of the respective segment, which may allow stacking of the tubes in the slots.

Referring again to FIG. 23, body 412 may be formed from a discontinuous annular member or open ring so that segments 412a and 412e may be separated to allow device 410 to be mounted about a pole, such as an IV pole. Therefore, the diameter of the central opening 418a may be in a range of about 0.5 to 1.0 inch or in a range of 0.75 to 0.90 inches. To facilitate the separation of the two end segments of the device, segments 412a and 412e may include angled surfaces 412a′ and 412e′, which can form cam surfaces so that when the device is pressed against a pole, the pole will press against the angle surface and thereby generate separation forces to separate the two end segments of the device and allow the pole to be inserted into central opening 418a. Central opening 418 may be sized to be slightly smaller than the pole diameter so that when the pole is inserted into the central opening the two end segments of the body will be separated. Further, device 410 may made from a material with relatively high friction and sufficient resiliency so that when device 410 is mounted about the pole, body 412 will generate a spring force to frictionally couple device 410 to the pole.

The above description is that of several embodiments of the invention. Various alterations and changes can be made from these embodiments without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

We claim:

1. A line management device comprising:
a body with a slot for receiving a tube, said slot having a shape; and
a base adapted to mount said body to a support surface, said body configured to deflect or deform relative to said base when subject to a load when said base is mounted to a surface but without affecting said shape of said slot.

2. The line management device according to claim 1, wherein said body is formed from foam.

3. The line management device according to claim 1, wherein said body includes a plurality of said slots.

4. The line management device according to claim 3, wherein one or more of said slots are color coded for a specific use.

5. The line management device according to claim 3, wherein said body has a curved outer surface, said slots extending into said curved outer surface.

6. The line management device according to claim 3, wherein each slot has longitudinal extent and a transverse extent generally perpendicular to its longitudinal extent, said transverse extent of at least one slot being different than said transverse extent of the other slots.

7. A line management device comprising:
a body with a slot for receiving a tube; and
a base adapted to mount said body to a support surface, and said base being configured to adapt to a non-planar surface without affecting the shape of the slot.

8. The line management device according claim 7, wherein said body is formed from foam.

9. The line management device according to claim 7, wherein said body includes a plurality of said slots.

10. The line management device according to claim 9, wherein one or more of said slots may be color coded for a specific use.

11. The line management device according to claim 7, wherein said body has a curved outer surface, the slot extending into the curved outer surface.

12. The line management device according to claim 9, wherein said body has a plurality of curved outer surfaces, said slots extending between said curved outer surfaces.

13. The line management device according to claim 12, wherein said plurality of slots comprises a first plurality of slots, said body including a second plurality of slots, said second plurality of slots extending into said curved outer surfaces.

14. The line management device according to claim 9, wherein said base comprises an annular portion, said body further including a plurality of segments extending from said annular portion, said slots extending between said segments.

15. The line management device according to claim 14, wherein said plurality of slots comprises a first plurality of slots, said body including a second plurality of slots, said second plurality of slots extending into said segments.

16. The line management device according to claim 15, wherein each of said segments has a curved outer surface.

17. The line management device according to claim 15, wherein each slot has longitudinal extent and a transverse extent generally perpendicular to its longitudinal extent, said longitudinal extents of said second plurality of slots being less than said longitudinal extents of said second plurality of slots.

18. The line management device according to claim 15, wherein each slot has longitudinal extent and a transverse extent generally perpendicular to its longitudinal extent, said transverse extents of said second plurality of slots being less than said transverse extents of said second plurality of slots.

19. The line management device according to claim 7, wherein said base includes a generally central passageway sized for receiving a pole.
